(19) United States
(12) Patent Application Publication
(10) Pub. No.: US 2002/0150582 A1
(43) Pub. Date: Oct. 17, 2002

(54) Method of treating or inhibiting cellular injury or cell death

(76) Inventors: Gregory S. Friedrichs, Yardley, PA (US); Robert E. Swillo, Ambler, PA (US); Brian H. Jow, Pennington, NJ (US); Terry R. Bridal, Doylestown, PA (US); Randal E. Numann, San Diego, CA (US); Linda M. Warner, West Chester, PA (US); Loran M. Killar, Newtown, PA (US)

Correspondence Address: Egon E. Berg, American Home Products Corporation, Patent Law Department - 2B, One Campus Drive, Parsippany, NJ, 07054, US

(21) Appl. No.: 09/779,039

(22) Filed: Feb. 8, 2001

(51) Int. Cl. A61K 39/395

(52) U.S. Cl. 424/178.1; 514/12; 424/192.1; 530/387.3

(57) ABSTRACT

This invention provides a method of treating or inhibiting cellular injury or cell death following an ischemic event, treating or inhibiting reperfusion injury, and reducing mortality following a myocardial infarction by providing therapy with a TNFα antagonist.

### CROSS REFERENCE TO RELATED APPLICATIONS

This application claims the benefit of U.S. Provisional Application No. (not yet known) which was converted from U.S. application Ser. No. 09/501,862 filed Feb. 10, 2000.

This invention relates to treating or inhibiting cellular injury or cell death following an ischemic event, treating or inhibiting reperfusion injury, and reducing mortality following a myocardial infarction by providing therapy with a TNFα antagonist.

The reduction or cessation of blood flow to a vascular bed accounts for a variety of clinical events that require immediate intervention and restitution of adequate perfusion to the jeopardized organ or tissue. Different tissues can withstand differing degrees of ischemic injury. However, all tissues will progress to irreversible injury and cellular necrosis if not reperfused. Impaired perfusion of cardiac tissue (ischemia) results in a loss of the heart's ability to function properly as the tissue becomes oxygen and energy deprived. Permanent injury is directly related to the duration of the oxygen deficit the myocardium experiences. Reperfusion of ischemic tissue simply refers to the restoration of flow to that tissue or organ system. The necessity of reperfusion, achieved by mechanical or pharmacological means has been accepted by the medical community, especially in the clinical setting of a myocardial infarction. Data suggests that “reperfusion injury” compromises the degree of tissue salvage when blood flow returns to the tissue.

Therapeutic interventions such as coronary angioplasty and thrombolytic therapy are directed toward the treatment of acute myocardial ischemia. It is well recognized that mortality among patients who are experiencing a myocardial infarction is dependent upon the extent of left ventricular dysfunction, which, in turn is directly related to the amount of myocardium that becomes infarcted and thus nonfunctional. There is general agreement that myocardial tissue subjected to an ischemic interval is dependent upon the restoration of blood flow within a defined period for cellular viability and function to be restored.

Compromised tissue following ischemia can only be recovered by reperfusing it. Though the act of reperfusion can extend injury further. As investigators began to recognize this, studies were directed to explore the mechanisms responsible, as well as to develop potential therapies to suppress cellular damage associated with reperfusion injury. A number of cellular mechanisms are believed to be responsible for ischemia-induced reperfusion injury.

TNFα is a cytokine secreted by macrophages and monocytes which causes a wide variety of effects on a number of cell types. TNF proteins initiate their biological effect on cells by binding to specific TNF receptor (TNFR) proteins expressed on the plasma membrane of a TNF responsive cell. The effects caused by TNFα include inhibitory or cytotoxic effects on tumor cell lines, stimulation of the proliferation of fibroblasts and the phaogcytic/cytotoxic activity of myeloid cells, induction of adhesion molecules in endothelial cells, inhibition of the synthesis of specific enzymes in adipocytes, and induction of the expression of histocompatibility antigens. [see, U.S. Pat. No. 5,610,279]. TNFα also causes pro-inflammatory actions which result in tissue injury, such as degradation of cartilage and bone [Saklatvala, Nature 322: 547 (1986); Bertolini, Nature 319: 516 (1986)]. TNFo: is also associated with infections, immune disorders, neoplastic pathologies, autoimmune pathologies, and graft vs. host disease. TNFα is also implicated in causing a wasting syndrome known as cachexia associated with cancer, which includes progressive weight loss, anorexia, and persistent erosion of lean body mass in response to malignant growth. [see WO 98/51344].

TNFα is also believed to contribute to the induction of ventricular dysfunction, pulmonary edema, and cardiomyopathy. [Torre-Amione G, J Am Coll Cardiol 27:1201-1206 (1996)] There is a growing body of evidence suggesting that components of the inflammatory cascade triggered by the binding of TNF to TNF receptor I and II (TNFR, p55, p75) are directly responsible for the acute deleterious effects observed in the myocardium [Oral, H., J Biol Chem. 272(8): 4836-4842 (1997); Kapadia, S., Am J. Physiol. 268: H517-H525 (1995)].

Inflammatory cytokines, including TNF, have been shown to be released by the myocytes immediately after the onset of ischemia [Meldrum, D. R., J Mol Cell Cardiol. 30:1683-1689 (1998)] and are believed to be involved in the expression of adhesion molecules that are instrumental in neutrophil extravasation. The sphingomyelinase pathway can be initiated by the release of TNF, and is considered the predominant signaling pathway of the cytokine [Kim et al., J Biol Chem 266: 484-489 (1991); Dressler et al., Science 255: 1715-1718 (1992); Yang, et al. J Biol Chem 268: 20520-20523 (1993)]. This pathway has been demonstrated in cardiac myocytes. [Oral et al., J Biol Chem 272: 4836-4842 (1997)]. Sphingomyelinases can be activated by TNF to breakdown the membrane bound sphingomyelin to ceramide. In turn, endogenous ceramidases catabolize ceramide to sphingosine. Both ceramide and sphingosine have been shown to possess second messenger properties. Sphingosine has been shown to depress cardiac function by decreasing calcium induced calcium release from the sarcoplasmic reticulum, as well as the ability to directly suppress L-type calcium current. Cain et al. [Crit Care Med. 27(7):1309-1318 (1999)] utilized stimulated human atrial trabeculae suspended in organ baths, and recorded the developed force the tissue generated. Graded concentrations of TNF-α, IL-1β or TNF-α+IL-1β were added and function was assessed. In addition, the tissues were exposed N-oleoyl ethanolamine (NOE) before TNF-α or IL-1β. TNF-α and IL-1β each depressed human myocardial function in a concentration-dependent fashion. Inhibition of myocardial sphingosine by NOE abolished the myocardial depressive effects of either TNF-α or IL-1β. The investigators concluded that TNF-α and IL-1β separately and synergistically depress human myocardial function. Sphingosine likely participates in the TNF-alpha and IL-1 beta signal leading to human myocardial functional depression.

Cell injury has also been demonstrated in other tissues. Adult human kidney proximal tubular (HK-2) cells were cultured for 0-20 hr in the presence or absence of sphingosine and metabolites as well as C2, C8, or C16 ceramide. Sphingosine (>or =10 microM), and selected ceramides (C2 and C8) each induced rapid, concentration- dependent cytotoxicity in the absence of DNA laddering or morphologic changes of apoptosis, suggesting a necrotic form of cell death. The investigators could reproduce the results in human foreskin fibroblasts, suggesting broad-based relevance to the area of acute cell injury and repair [Iwata et al., PNAS 92(19):8970-8974 (1995)].

ENBREL (etanercept; p75TNFR:Fc) is a dimeric fusion protein consisting of the extracellular ligand-binding protein of the human 75 kilodalton (p75) tumor necrosis factor receptor (TNFR) linked to the Fc portion of human IgG1. Etanercept is a TNFα antagonist currently marketed for the treatment of rheumatoid arthritis, and is undergoing clinical trials for treatment of chronic heart failure [Bozkurt B, JACC (Suppl) 184-185A (1999); Deswal A, Circulation (suppl) 96(8):I-323 (1997)].

WO 98/51344 discloses the use of a TNF(X antagonist in combination with a VEGF antagonist for the treatment or prevention of TNF-mediated diseases including rheumatoid arthritis, Crohn's disease, and acute and chronic immune diseases associated with transplantation.

### DESCRIPTION OF THE INVENTION

This invention provides a method of treating or inhibiting cellular injury or cell death following an ischemic event which comprises providing an effective amount of a TNFα antagonist. More particularly, this invention provides a method of treating or inhibiting cellular injury or cell death resulting from myocardial infarction, myocardial ischemia, retinal ischemia, central retinal occlusion, peripheral arterial occlusion (i.e., an embolism), transient ischemic attacks (i.e., ceberal ischemic attacks), ischemic stroke, ischemic arterial obstruction, reperfusion injury resulting from frostbite, arterial thrombosis and occlusion, and crush injury by providing an effective amount of a TNFα antagonist. This invention also provides a method of reducing mortality following myocardial infarction by providing an effective amount of a TNFα antagonist. This invention additionally provides a method of inhibiting cardiac damage following a cardiac ischemic event by providing an effective amount of a TNFα antagonist. This invention further provides a method of treating or inhibiting reperfusion injury by providing an effective amount of a TNFα antagonist.

As used in accordance with this invention, the term providing an effective amount of a TNFα antagonist means either directly administering such antagonist, or administering a prodrug, derivative, or analog which will form an effective amount of the antagonist within the body.

The term TNFα antagonist has been well defined in WO 98/51344, and is defined as decreases, blocks, inhibits, abrogates or interferes with TNFα activity in vivo. For example, a suitable TNFα antagonist can bind TNFα and includes anti-TNFα antibodies, antigen-binding fragments thereof, and receptor molecules and derivatives which bind specifically to TNFα. A suitable TNFα antagonist can also prevent or inhibit TNFα synthesis and/or TNFα release and includes compounds such as thalidomide, tenidap, and phosphodiesterase inhibitors, such as, but not limited to, pentoxifylline and rolipram. A suitable TNFα antagonist that can prevent or inhibit TNFα synthesis and/or TNFα release also includes A2b adenosine receptor enhancers and A2b adenosine receptor agonists (e.g., 51-(N-cyclopropyl)-carboxamidoadenosine, 51-N-ethylcarboxamidoadenosine, cyclohexyladenosine and R-N6-phenyl-2-propyladenosine) See, for example, Jacobson, GB 2 289 218A. A suitable TNFα antagonist can also prevent or inhibit TNFα receptor signalling and includes mitogen activated protein (MAP) kinase inhibitors. Other suitable TNFα antagonists include agents which decrease, block, inhibit, abrogate or interfere with membrane TNFα cleavage, such as, but not limited to, metalloproteinase inhibitors; agents which decrease, block, inhibit, abrogate or interfere with TNFα activity, such as, but not limited to, angiotensin converting enzyme (ACE) inhibitors, such as captopril, enalapril and lisinopril; and agents which decrease, block, inhibit, abrogate or interfere with TNFα production and/or synthesis, such as, but not limited to, MAP kinase inhibitors.

It is preferred that the TNFα antagonist is a TNF receptor molecule that binds TNFα. It is more preferred that the TNF receptor molecule is a TNF receptor fragment/immunoglobulin fusion protein. It is still more preferred that the fusion protein comprises a fragment of TNFR and a portion or the entire constant region of a human immunoglobulin heavy chain.

A particularly preferred TNFα antagonist is etanercept (p75TNFR:Fc), which is a dimeric fusion protein consisting of the extracellular ligand-binding protein of the human 75 kilodalton (p75) tumor necrosis factor receptor (TNFR) linked to the Fc portion of human IgG1. Etanercept is commercially available as ENBREL, and is currently approved for use in treating rheumatoid arthritis. Etanercept can be prepared according to the procedures described in U.S. Pat. Nos. 5,605,690, 5,478,925, EP 464533, and EP670730, which are hereby incorporated by reference.

Another preferred TNFα antagonist is designated as p55TNFR:Fc, which is a dimeric fusion protein consisting of the extracellular ligand-binding protein of the human 55 kilodalton (p55) tumor necrosis factor receptor (TNFR) linked to the Fc portion of human IgG1. The production of p55TNFR:Fc is disclosed in U.S. Pat. No. 5,610,279, which is incorporated by reference.

The ability of TNFα antagonists to treat or inhibit cellular injury or cell death following an ischemic event and to treat or inhibit reperfusion injury was evaluated in two in vivo standard pharmacological test procedures. The first test procedure evaluated the effects of TNF and sphingosine on cardiac function, and the second test procedure evaluated the survival after a 30 minute occlusion of the main coronary artery followed by reperfusion. Etanercept was evaluated as a representative TNFα antagonist in the second test procedure which emulates an acute myocardial infarction. An in vitro standard pharmacological test procedure was also performed to evaluate the cardiodepressant effect of sphingosine on myocytes. The procedures used and results obtained are described below.

Procedures

Surgical Preparation.

Male Sprague-Dawley rats weighing 505±5 g were anesthetized with sodium pentobarbital (50 mg/kg I.P.). An endotracheal tube was secured in place and connected to a small-animal respirator (Harvard Apparatus, Model 683, South Natick, Mass.) set on 100 breaths/min, with a tidal volume of 2-3 mnL/breath. Body temperature was maintained using a heating pad with circulating warm water (K-model 100, Baxter Laboratories). A left thoracotomy was performed, the heart exposed and the pericardium removed. Left ventricular pressure was measured using a saline-filled polyethylene catheter attached to an angiocatheter (20 Gauge) inserted through the apex of the heart and connected to a P23 ID Statham/Gould pressure transducer. Arterial pressure was monitored via a saline-filled polyethylene catheter (PE 50) introduced into the left carotid artery. The right jugular vein was also cannulated with a polyethylene catheter (PE 50) for IV drug infusions and volume repletion (0.9% NaCl). Subcutaneous needles were positioned in the limbs for ECG recordings. All data outputs were recorded on a Gould Model 6600 (Valley View, Ohio) series recorder with a Po-Ne-Mah data acquisition system (Valley View, Ohio) and displayed on a physiology platform CRS800W/CRS400W recorder (General Scanning Inc., Bedford Mass.).

In the first test procedure, the rats were surgically prepared (open chest, but no occlusion) and were administered TNF, sphingomyelin, or sphingosine by slow i.v. infusion (0.1 mg/kg over 5 minutes). Each animal was observed continuously for 15 minutes, whereupon a second does was administered by slow i.v. infusion (0.3 mg/kg over 5 minutes) and each animal was again observed for 15 minutes.

In the second test procedure, the main coronary artery of rats undergoing coronary occlusion was located and occluded close to its origin using a 5-0 suture passed underneath the vessel that could be tightened over a short section of PE tubing (PE 20) to initiate regional ischemia. Reperfusion was reinstated by removing the short segment of PE tubing.

Determination of Infarct Size.

The heart was removed and sliced in five to six coronal sections, which were immersed in 1% triphenyltetrazolium chloride (TTC) for 10-15 minutes. The heart sections were removed, blotted dry and traced onto acetate sheets. The areas of infarction were clearly demarcated by a pale appearance in the ischemic zone, and a brick red color of non-infarcted myocardium. The areas of all sections were determined by planimetric methods and infarct area was expressed as a percentage of the left ventricle.

TNF Determination in Rat Serum.

Blood samples were collected in syringes in the absence of any anticoagulant and immediately placed in Microtainer® serum separator tubes (Becton Dickinson) and centrifuged at 2000 g for 6 minutes. The serum was removed, frozen immediately, and stored at −20° C. until the analysis could be performed. The concentration of serum TNF collected at preselected time points was determined for individual rats by enzyme-linked immunosorbent assay (ELISA), utilizing the Factor-Test-X (Genzyme Inc, Cambridge, Mass., Cat #80-3905-01) according to the manufacturer's instructions. Briefly, 100 μL of serum was diluted 1:2 in 0.1% bovine serum albumin/phosphate buffered saline and added in duplicate to a 96-well microtiter plate. A standard curve was generated by plotting the concentrations of rat TNF standards versus their absorbances. The manufacturer of the assay and validation in our laboratory have indicated that this assay is able to detect both free TNF and TNF bound to etanercept (data not shown). The detection limit of this assay was 10 pg/mL. Additionally, the manufacturer has determined that this ELISA is highly specific for rat TNF. Concentrations that reached 10⁶ pg/mL of rat IFN-γ, GRO-β/MIP-2, GRO/KC, and interleukins IL-1β, IL-2, and IL-4, as well as mouse LIF, SCF, GM-CSF, and interleukins IL-1α, IL-3, IL-5, IL-6, IL-7, and IL-10, did not yield detectable cross reactivity.

Ventricular Myocyte Isolation.

Ventricular myocytes were isolated using a modified Langendorff perfusion procedure outlined by Silver, et al., (13) Briefly, cats of either sex, weighing 2-4 kg were anaesthetized with pentobarbital sodium (40 mg/kg I.P.). Under anesthesia, a stemotomy was performed, the heart rapidly excised, and then immersed in Ca⁺⁺-free Krebs-Henseleit buffer solution (KHB) at 4° C. for aortic cannulation. KHB had the following composition (in mM): 130 NaCl, 4.8 KCl, 1.2 MgSO₄, 1.2 NaH₂PO₄, 25 NaHCO₃, 12.5 dextrose. Solution pH was 7.35-7.40 when equilibrated with 95% O₂/5% CO₂ gas mixture. The solution was actively aerated throughout the procedure. The cannulated heart was rinsed with KHB at 37° C. for 2-4 minutes followed by perfusion for 12-15 minutes with KHB containing 0.7 mg/mL Type II collagenase (197 U/mg; Worthington; Freehold, N.J. USA). Digested ventricular tissue was then dissected from the atria, minced, and filtered through a 200 μm pore nylon mesh. Filtrate was centrifuged at 50× g for 1-2 minutes and the separated cell pellet was resuspended in fresh KHB. The latter process was performed three times. On the third iteration, the pellet was resuspended with KHB containing 2% bovine serum albumin and 100 μM Ca⁺⁺. The resulting cell suspension was divided into two aliquots. One was diluted 1:1 with Tyrode's solution (composition below), maintained at room temperature (19-25° C.), and was used for cellular recordings within 12 hours of isolation. The second aliquot was utilized to plate cells used for subsequent recordings and was dispensed into a 1:1 mix of DMEM/F-12 culture media (Bio Whittaker, Walkersville, Md., USA), supplemented with streptomycin sulfate (200 μg/mL) and penicillin-G sodium salt (200 units/mL). Plated cells were maintained at room temperature in an incubator (pH=7.2). The suspension media was changed every two days.

Myocyte Electrophysiological Recording.

Patch-clamp current and voltage recordings were made with the ruptured patch whole-cell configuration at 36-37° C. (14). For studies on L-type Ca²⁺ currents, cells were bathed with modified Tyrode's containing (in mM): 157 TEA-Cl, 5 CaCl₂, 0.5 MgCl₂, and 10 HEPES. Electrode internal solution contained (in mM): 10 L-Glutamic Acid, 20 CsCl, 10 EGTA, 1 MgCl₂, 1 CaCl₂, 20 HEPES, and 5 ATP-Mg2; pH adjusted with CsOH. In all of the studies, electrode resistance was measured to be 2-3.5 MΩ. The reference zero potential was adjusted in the bath before forming seals. Recordings were performed with an Axon Instruments 200B amplifier (Axon Instruments, Culver City, Calif. USA) interfacing a DigiData 1200 DA/AD acquisition system. Ionic currents were evoked by depolarizing voltage step (1 sec-long) from −30 to +60 mV, in 10-mV increments, from a holding potential of −40 mV delivered at the frequency of 1 Hz. Action potentials were elicited at a frequency of 1 Hz by injection of brief depolarizing current pulses. Software used in data acquisition and analysis was pClamp v.6.04 and Origin v 5.0 (Microcal Software, Northampton, Mass.).

Results

Hemodynamic Parameters in Non-Occluded Rats.

The following table shows the effects of sphingosine, sphingomyelin, and TNF on the following hemodynamic parameters in open chest rats in the absence of myocardial ischemia: heart rate, mean blood pressure, left ventricular blood pressure (LVL) and its first derivative (+dP/dt).

### Cardiovascular Parameters Measured in Open Chest Rats in the Absence of Myocardial Ischemia

| GROUPS | Heart Rate | Mean BP | LVPdev | +dP/dt |
|---|---|---|---|---|
| Rat TNF-α | | | | |
| 0.1 mg/kg iv (n = 5) + | −3 ± 1 | 4 ± 7 | 1 ± 4 | 10 ± 5 |
| 0.3 mg/kg iv | −3 ± 2 | 4 ± 6 | −1 ± 3 | 9 ± 8 |
| Sphingomyelin | | | | |
| 0.1 mg/kg iv (n = 4) + | 7 ± 9 | −3 ± 17 | 3 ± 11 | 1 ± 13 |
| 0.3 mg/kg iv | 8 ± 10 | 2 ± 2 | 4 ± 12 | 6 ± 15 |
| Sphingosine | | | | |
| 0.3 mg/kg iv (n = 4) + | −4 ± 4 | −24 ± 12 | −24 ± 8* | −33 ± 12* |
| 1.0 mg/kg iv | −18 ± 1* | −17 ± 19 | −24 ± 14 | −32 ± 18* |

Data are expressed as mean ± sem. Data are percent change from pre-drug baseline 15 minutes after drug administration. * indicates p<0.05 vs other pretreatment groups over the entire observation period.

The results of this test procedure show that following infusion of sphingosine (0.1+0.3 mg/kg), overall myocardial function was significantly depressed: LVP was depressed by 24±8% and +dP/dt was reduced 33±12% from baseline. The dose of TNF that was administered exogenously to the rats in this test procedure resulted in serum concentrations two-fold higher than the Cₘₐₓ of TNF generated endogenously after myocardial ischemia. However, even very high serum concentrations of TNF failed to produce acute cardiodepression in the absence of an inflammatory response, which is necessary for initiation of the sphingolipid cascade. In the absence of myocyte injury, one would not expect sphingomyelin administration to depress function since membrane sphingomyelinases would not have been stimulated by increased concentrations of TNF to degrade sphingomyelin to ceramide, the metabolite previously shown to decrease contractility in isolated myocytes.

Effects of Sphingosine on Isolated Myocytes.

To further evaluate the effects of sphingosine on cardiac function and cellular injury, isolated myocytes were isolated and the effects of sphingosine on calcium currents were measured as described above. Previous studies have shown that sphingosine can effect the electrogenesis of the action potential by decreasing Ca⁺² release from the sarcoplasmic reticulum [Yasui, et al. Am J. Physiol. 270: C645-C649 (1996); MacDonell, et al. Am J Physiol. 275: H2291-H2299 (1998)]. The results obtained in the standard =pharmacological test procedure described above showed that sphingosine shortened =the action potential duration (APD) of isolated feline ventricular myocytes in a concentration-dependent manner, with 0.25, 2.5 and 25 μM sphingosine reducing the duration at 95% of full repolarization (APD₉₅) by 16±2%, 28±2%, and 39±2% (n=4), respectively. The shortening of APD was mostly the result of a depression of the plateau phase. The Ca⁺² current (I_{Ca-L}) was isolated by suppressing other K⁺ currents by using Cs⁺, a blocker of K⁺ currents, in both the external and in the pipette solution. The fast Na⁺ current (I_{Na}) was eliminated by holding the myocytes at −40 mV, a potential where (I_{Na}) is largely inactivated, and by substituting NaCl with TEA-Cl in the external solution. Exposure to sphingosine (2.5 and 25 μM) caused a significant block of I_{ca-L} (17±7 and 75±4% block at 25 μM). The shortening of the cardiac action potential and reducing the inward Ca⁺² current would be expected to correlate with the negative inotropic changes seen directly following systemic administration or, indirectly, following activation of TNFR1. Ultimately, sphingosine (25 μM) lead to myocardial cell death assessed by its resulting morphology and lack of viability soon after exposure to sphingosine at the highest concentration. Treatment of myocytes with TNF (200-20,000 U/mL) did not alter action potential or I_{Ca+2-L}.

TNF Levels in Occluded Rats.

As described above, rats were surgically prepared and the coronary artery was occluded. The following table shows the serum concentration of TNF in open chest rats undergoing myocardial ischemia.

| GROUPS | Vehicle | Etanercept |
|---|---|---|
| −30 min | 0 ± 0 | 0 ± 0 |
| 0 min | 6056 ± 925 | 2299 ± 357* |
| 30 min | 2122 ± 371 | 2057 ± 349 |
| 60 min | 740 ± 153 | 1592 ± 258* |
| 90 min | 585 ± 112 | 1108 ± 279* |
| 120 min | 338 ± 44 | 721 ± 209 |
| 150 min | 204 ± 7 | 580 ± 193* |

Data are expressed as mean ± sem in units of pg/ml.
*indicates p < 0.05 vs the vehicle treatment group at the identical timepoint.
Baseline = −30 min,
End of Ischemia = 0 min, and Reperfusion = 30, 60, 90 . . . min.

In this test procedure, rats that had their chests surgically opened, but did not have any induced occlusion (sham-operated animals) had a stable TNFα concentration throughout the duration of the procedure, with a maximum concentration of 242±90 pg/mL. These data show that vascular occlusion produced vastly increased levels of TNFα, which peaked at the conclusion of the ischernic period. The results also showed that etanercept significantly reduced the massive TNFα spike in concentration in response to vascular occlusion at 0 minutes.

Effect of TNFα Antagonist Treatment on Mortality in Occluded Rats.

The percent survival following myocardial ischemia/reperfusion was also evaluated in this test procedure. The results obtained are summarized in the table below.

### Percent Survival of Open Chest Rats Undergoing Myocardial Ischemia/Reperfusion in the Presence or Absence of Etanercept (3 mg/kg iv)

| GROUPS | Vehicle | Etanercept |
|---|---|---|
| −30 min | 100 | 100 |
| 0 min | 100 | 100 |
| 30 min | 83 | 100 |
| 60 min | 66 | 100 |
| 90 min | 50 | 89 |
| 120 min | 33 | 89* |
| 150 min | 17 | 88* |

Data are expressed as percent survival.
*indicates p < 0.05 vs the vehicle treatment group at the identical timepoint.
Baseline = −30 min,
End of Ischemia = 0 min, and Reperfusion = 30, 60, 90 . . . min.

In this standard pharmacological test procedure, etanercept (3 mg/kg i.v.) administered immediately before occlusion significantly reduced mortality resulting from the myocardial ischemia/reperfusion. During the latter stages of reperfusion (t=90 minutes) a difference began to emerge with regard to overall mortality. For example, 4 out of 9 rats were dead at t=90 minutes in the vehicle treated group compared to 1 out of 9 in the etanercept treated group, though the difference failed to reach statistical significance (p=0.08). The difference between the etanercept and vehicle-control groups achieved statistical significance at 120 and 150 min of reperfusion. Of the seven deaths observed in the vehicle-treated group, six were due to acute pump failure and progressive bradycardia, and one animal died of ventricular arrhythmias early after reperfusion. The two deaths in the etanercept treated group were both due to bradycardia and pump failure. Infarct size, expressed as a percentage of the left ventricle, was 24±3% for Etanercept and 26±2% for the vehicle-control group, showing that the difference of survival was not the result of unequal infarct size. These results demonstrate that treatment with a TNFα antagonist reduced the mortality resulting from myocardial ischemia/reperfusion, presumably by preventing the cascade generating sphingosine from sphingomyelin which follows TNFα binding to TNFR1 in response to the ischemic/reperfusion injury.

Based on the results obtained in the standard pharmacological test procedures described above, TNFα antagonists are useful in reducing mortality following myocardial infarction. Based on the results obtained, TNFα antagonists are also useful in treating or inhibiting cellular injury or cell death following an ischemic event. More particularly, this invention provides a method of treating or inhibiting cellular injury or cell death resulting from myocardial infarction, myocardial ischemia, retinal ischemia, central retinal occlusion, peripheral arterial occlusion (i.e., an embolism), transient ischemic attacks (i.e., ceberal ischemic attacks), ischemic stroke, ischemic arterial obstruction, injury resulting from frostbite, arterial thrombosis and occlusion, and crush injury. This invention is also useful in treating or inhibiting reperfusion injury. Treatment with a TNFα antagonist will also be useful prior to or during procedures which involve ischemic events followed by reperfusion, such as transplant surgery, when the donor organ undergoes a period of ischemia, and is then reperfused by the recipients blood supply; angioplasty or coronary stent placement; thrombolytic therapy; heart valve replacement; and bypass surgery.

TNFα antagonists may be formulated neat or may be combined with one or more pharmaceutically acceptable carriers for administration according to standard method for the formulation of pharmaceutical agents. Routes of administration include oral, parenteral (including, for example, intravenous, intramuscular injection, subcutaneous injection), intranasal, intraperitoneal, rectal, vaginal, and transdermal. The routes of administration vary with the nature of the TNFα antagonist and reason for administration. For example, where the TNFα antagonist will rapidly degrade in the gut, administration is preferably made parenterally. It is preferable to provide etanercept intravenously for the treatment or inhibition of cellular injury or cell death following an ischemic event, because of the acid labile nature of etanercept and the necessity for rapid onset of action.

When the TNFα antagonist is to be provided orally, it can be provided in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

Formulation for tablet or capsule administration may include solid carriers including starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

When the TNFα antagonist is to be administered parenterally or intraperitoneally, solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils. Etanercept, for example is commercially available as a white, preservative free, lyophilized powder for parenteral administration after reconstitution with water.

It is anticipated that the dosage the TNFα antagonist will vary according to the nature of the TNFα antagonist, the reason for administration, and individual patient receiving therapy. For chronic therapy, it is generally recommended that treatment begin with the smallest effective dosage, with dosage adjustments being made through physician monitoring. For treatment with etanercept, projected intravenous dosage would be between 0.05-25 mg/kg etanercept. It is contemplated that the TNFc antagonist may be administered in a single dose or over several doses in response to a particular ischemic event, or may be administered chronically to inhibit cellular damage or death in response to future ischemic events. For example, it is anticipated that a TNFα antagonist may be administered chronically to a patient suffering from transient ischemic events, which often occur over long periods of time. Alternatively, it is also contemplated that the TNFα antagonist may be administered prophylactically in situations where it is anticipated that an ischemic event will occur (for example, prior to a transplant procedure or angioplasty procedure).

1. A method of treating or inhibiting cellular injury or inhibiting cell death following an ischemic event in a mammal in need thereof, which comprises providing an effective amount of a TNF(X antagonist to said mammal.

2. The method according to claim 1, wherein the cellular injury or death results from myocardial infarction, myocardial ischemia, retinal ischemia, central retinal occlusion, peripheral arterial occlusion, transient ischemic attacks, ischemic stroke, ischemic arterial obstruction, frostbite, arterial thrombosis and occlusion, or crush injury.

3. The method according to claim 1, wherein the TNFα antagonist is a TNF receptor/immunoglobulin fusion protein.

4. The method according to claim 3, wherein the TNFa antagonist comprises a fragment of TNFR and a portion or the entire constant region of a human immunoglobulin heavy chain.

5. The method according to claim 4, wherein the TNFA antagonist is etanercept.

6. The method according to claim 4, wherein the TNFα antagonist is p55TNFR:Fc.

7. A method of treating or inhibiting reperfusion injury in a mammal in need thereof, which comprises providing an effective amount of a TNF(X antagonist to said mammal.

8. The method according to claim 7, wherein the injury results from transplant surgery, angioplasty, coronary stent placement, thrombolytic therapy, heart valve replacement, or bypass surgery.

9. The method according to claim 7, wherein the TNFα antagonist is a TNF receptor/immunoglobulin fusion protein.

10. The method according to claim 9, wherein the TNFα antagonist comprises a fragment of TNFR and a portion or the entire constant region of a human immunoglobulin heavy chain.

11. The method according to claim 10, wherein the TNFα antagonist is etanercept.

12. The method according to claim 10, wherein the TNFα antagonist is p55TNFR:Fc.

13. A method of reducing mortality following a myocardial infarction in a mammal in need thereof, which comprises providing an effective amount of a TNFα antagonist to said mammal.

14. The method according to claim 13, wherein the TNFα antagonist is a TNF receptor/immunoglobulin fusion protein.

15. The method according to claim 14, wherein the TNFα antagonist comprises a fragment of TNFR and a portion or the entire constant region of a human immunoglobulin heavy chain.

16. The method according to claim 15, wherein the TNα antagonist is etanercept.

17. The method according to claim 15, wherein the TNFα antagonist is p55TNFR:Fc.
